SEQUENCE LISTING (6 sequences)

SEQ ID NO: 1 - protein, 397 aa, Xanthomonas oryzae pathovar oryzae
APARGTLLTSNFLTSYTRDAISAMLASGSQPASGSQPEQAKCNVRVAEFTYATIGVEGEPATASGVLLIPGGERCSGPYPLLGWGHPTEALRAQEQAKEIRDAKGDDPLVTRLASQGYVVVGSDYLGLGKSNYAYHPYLHSASEASATIDAMRAARSVLQHLKTPLSGKVMLSGYSQGGHTAMATQREIEAHLSKEFHLVASAPISGPYALEQTFLDSWSGSNAVGENTFGILLGSYAIVAMQHTYKNIYLEPGQVFQDPWAAKVEPLFPGKQSLTDMFLNDTLPSIDKVKSYFQPGFYSDFPSNPANPFRQDLARNNLLEWAPQTPTLLCGSSNDATVPLKNAQTAIASFQQRGSNQVALVDTGTGNASDNSAFAHMLTKESCIVVVRDQLLDKQR

SEQ ID NO: 2 - protein, 397 aa, Xanthomonas oryzae pathovar oryzae
APARGTLLTSNFLTSYTRDAISAMLASGSQPASGSQPEQAKCNVRVAEFTYATIGVEGEPATASGVLLIPGGERCSGPYPLLGWGHPTEALRAQEQAKEIRDAKGDDPLVTRLASQGYVVVGSDYLGLGKSNYAYHPYLHSASEASATIDAMRAARSVLQHLKTPLSGKVMLSGYSQGGHTAMATQREIEAHLSKEFHLVASAPISGPYALEQTFLDSWSGSNAVGEWTFGILLGSYAIVAMQHTYKNIYLEPGQVFQDPWAAKVEPLFPGKQSLTDMFLNDTLPSIDKVKSYFQPGFYSDFPSNPANPFRQDLARNNLLEWAPQTPTLLCGSSNDATVPLKNAQTAIASFQQRGSNQVALVDTGTGNASDNSAFAHMLTKESCIVVVRDQLLDKQR

SEQ ID NO: 3 - protein, 397 aa, Xanthomonas oryzae pathovar oryzae
APARGTLLTSNFLTSYTRDAISAMLASGSQPASGSQPEQAKCNVRVAEFTYATIGVEGEPATASGVLLIPGGERCSGPYPLLGWGHPTEALRAQEQAKEIRDAKGDDPLVTRLASQGYVVVGSDYLGLGKSNYAYHPYLHSASEASATIDAMRAARSVLQHLKTPLSGKVMLSGYSQGGHTAMATQREIEAHLSKEFHLVASAPISGPYALEQTFLDSWSGSNAVGENTFAILLGSYAIVAMQHTYKNIYLEPGQVFQDPWAAKVEPLFPGKQSLTDMFLNDTLPSIDKVKSYFQPGFYSDFPSNPANPFRQDLARNNLLEWAPQTPTLLCGSSNDATVPLKNAQTAIASFQQRGSNQVALVDTGTGNASDNSAFAHMLTKESCIVVVRDQLLDKQR

SEQ ID NO: 4 - protein, 397 aa, Xanthomonas oryzae pathovar oryzae
APARGTLLTSNFLTSYTRDAISAMLASGSQPASGSQPEQAKCNVRVAEFTYATIGVEGEPATASGVLLIPGGERCSGPYPLLGWGHPTEALRAQEQAKEIRDAKGDDPLVTRLASQGYVVVGSDYLGLGKSNYAYHPYLHSASEASATIDAMRAARSVLQHLKTPLSGKVMLSGYSQGGHTAMATQREIEAHLSKEFHLVASAPISGPYALEQTFLDSWSGSNAVGENTFIILLGSYAIVAMQHTYKNIYLEPGQVFQDPWAAKVEPLFPGKQSLTDMFLNDTLPSIDKVKSYFQPGFYSDFPSNPANPFRQDLARNNLLEWAPQTPTLLCGSSNDATVPLKNAQTAIASFQQRGSNQVALVDTGTGNASDNSAFAHMLTKESCIVVVRDQLLDKQR

SEQ ID NO: 5 - protein, 397 aa, Xanthomonas oryzae pathovar oryzae
APARGTLLTSNFLTSYTRDAISAMLASGSQPASGSQPEQAKCNVRVAEFTYATIGVEGEPATASGVLLIPGGERCSGPYPLLGWGHPTEALRAQEQAKEIRDAKGDDPLVTRLASQGYVVVGSDYLGLGKSNYAYHPYLHSASEASATIDAMRAARSVLQHLKTPLSGKVMLSGYSQGGHTAMATQREIEAHLSKEFHLVASAPISGPYALEQTFLDSWSGSNAVGENTFFILLGSYAIVAMQHTYKNIYLEPGQVFQDPWAAKVEPLFPGKQSLTDMFLNDTLPSIDKVKSYFQPGFYSDFPSNPANPFRQDLARNNLLEWAPQTPTLLCGSSNDATVPLKNAQTAIASFQQRGSNQVALVDTGTGNASDNSAFAHMLTKESCIVVVRDQLLDKQR

SEQ ID NO: 6 - protein, 397 aa, Xanthomonas oryzae pathovar oryzae
APARGTLLTSNFLTSYTRDAISAMLASGSQPASGSQPEQAKCNVRVAEFTYATIGVEGEPATASGVLLIPGGERCSGPYPLLGWGHPTEALRAQEQAKEIRDAKGDDPLVTRLASQGYVVVGSDYLGLGKSNYAYHPYLHSASEASATIDAMRAARSVLQHLKTPLSGKVMLSGYSQGGHTAMATQREIEAHLSKEFHLVASAPISGPYALEQTFLDSWSISNAVGENTFGILLGSYAIVAMQHTYKNIYLEPGQVFQDPWAAKVEPLFPGKQSLTDMFLNDTLPSIDKVKSYFQPGFYSDFPSNPANPFRQDLARNNLLEWAPQTPTLLCGSSNDATVPLKNAQTAIASFQQRGSNQVALVDTGTGNASDNSAFAHMLTKESCIVVVRDQLLDKQR

(19) United States
(12) Patent Application Publication
(10) Pub. No.: US 2012/0190071 A1
(43) Pub. Date: Jul. 26, 2012

(54) LIPA AND ITS VARIANT USEFUL FOR BIOFUEL PRODUCTION

(75) Inventors: Aparna Gudlur, Hyderabad (IN); Ramesh Venkata Sonti, Hyderabad (IN); Rajan Sankaranarayanan, Hyderabad (IN)

(73) Assignee: COUNCIL OF SCIENTIFIC & INDUSTRIAL RESEARCH, New Delhi (IN)

(21) Appl. No.: 13/320,234

(22) PCT Filed: May 11, 2010
(86) PCT No.: PCT/IB10/01080

§ 371 Date: Apr. 4, 2012

(30) Foreign Application Priority Data
May 12, 2009 (IN) 970/DEL/2009

(51) Int. Cl. C12P 19/00; C12N 9/18

(52) U.S. Cl. 435/72; 435/197

(57) ABSTRACT

The present invention relates to LipA and its variant that binds to the glucosyl- and acyl chain- region of its glycoside substrate in plant cell walls useful for biofuel production, wherein presence of a hydrophobic pocket confers extensive hydrophobic interaction of the acyl chain with the rest of the tunnel residues.

### FIELD OF INVENTION

The present invention relates to a variant of LipA that binds to the glucosyl- and acyl chain-region of its glycoside substrate in plant cell walls useful for biofuel production. It further relates to an esterase enzyme LipA and its variants from Xanthomonas as identified by its novel mode of action as determined by crystal structural and functional analysis and the applications of this said mode of activity for improving the enzyme mixes used in biofuel production. More specifically, the invention relates to the plant cell wall degrading activity involving substrate recognition based on recognition of a glucosyl- and an acyl chain-binding region in a glycoside substrate in plant cell walls from Xanthomonas oryzae and its related homologs. This invention relates to a method for improving the said activity of the said enzyme deciphered with the help of the enzyme X-ray crystal structure.

### BACKGROUND OF INVENTION

Around 85% of the total energy requirement of the world is meted out by fossil fuels like coal, natural gas and crude oil. A limited source of supply and extensive pollution due to fossil fuels together with an ever-increasing demand for energy has fuelled the efforts to find alternative sources of energy.

Biofuel or fuel made from living organisms or from metabolic by-products like organic or food waste is coming forth as an important alternative source of energy. It is derived from biomass of recently living organisms. Wastes from industry, agriculture, forestry, and households including straw, lumber, manure, sewage, garbage and food leftovers can be degraded to produce biofuels (Marshall, A. T. 2007. Bioenergy from waste: a growing source of power, waste management. World Magazine. April: 34-37). The chemical energy in biofuels is stored in the form of carbon that was recently extracted from atmospheric carbon dioxide by growing plants, so burning it does not result in a net increase of carbon dioxide in the atmosphere. Therefore, biofuel is a non-polluting, renewable substitute for fossil fuels.

One commonly used strategy for fuel production from the plant biomass involves the degradation of complex polymers in the plant cell walls and the fermentation of constituent mixed sugars to butanol (replacement for gasoline), ethanol and methane (equivalent to biogas) (Doi, R. H., Kosugi, A., Murashima, K., Tamaru Y. and Han, S. O. 2003. Cellulosomes from mesophilic bacteria. Journal of Bacteriology. 185: 5907-5914). Cell walls, which comprise approximately 40-80% of the biomass of plants, are composed of an intricate network of cellulose-hemicellulose along with phenolics in the form of lignin and several mixed linkages (Knox, J. P. (2008) Revealing the structural and functional diversity of plant cell walls. Current Opinion Plant Biol. 11: 308-313). Grasses generally have a high biomass content, ˜50-60% of which can be converted to ethanol (Tilman, D., Hill, J. and Lehman, C. 2006. Carbon-negative biofuels from low-input high-diversity grassland biomass. Science. 314: 1598-1600).

The conversion of cellulose to fuel material is best achieved by using microbial cellulolytic enzymes. Bacterial cellulolytic machinery can be constructed in two ways, either by using independent extracellular cellulases that act synergistically to degrade cellulose or by using cellulosome complexes, which consists of a non-enzymatic scaffolding protein associated with various enzymatic subunits that act in concert to degrade cellulose and hemicelluloses (Himmel, M. E., Ding, S-Y., Johnson, D: K., Adney, W. S., Nimlos, M. R., Brady, J. W. and Foust. T. D. 2007. Biomass recalcitrance: engineering plants and enzymes for biofuels production. Science. 315: 804-807). However, degradation of only polysaccharide components is not enough for degradation of the whole biomass and the complex nature of the other constituents of the plant cell walls can hinder efficiency in the whole process (Himmel, M. E., Ding, S-Y., Johnson, D. K., Adney, W. S., Nimlos, M. R., Brady, J. W. and Foust. T. D. 2007. Biomass recalcitrance: engineering plants and enzymes for biofuels production. Science. 315: 804-807). Bacterial enzymes also increase the efficiency of plant cell wall degradation by proficient hydrolysis of the lignin content of the biomass (Duck, N. B., Carr, B., Koziel, M. G., Carozzi, N. and Vande, B. B. 2003. Methods for enzymatic hydrolysis of lignocellulose (WO/2003/093420). The use of xylanases, feruloyl esterases77, acetyl xylan esterases, pectin methyl esterases and proteases in conjunction with glucanases has been found to improve the process of biomass conversion (Berka, R., Cherry, J. 2005. Methods for degrading or converting plant cell wall polysaccharides (WO/2005/100582), West, S., Thomas, S., Connerton, I., Crepin, V., Faulds, C. B., Garcia-Conesa, M-T.,and Kroon, P. A. 2004. Feruloyl esterase and uses thereof (WO/2004/009804).

However, there are several crosslinks between hetero-polymers in the plant cell walls, which are not hydrolysed by the reported set of plant cell wall degrading enzymes. Therefore, there is a need to discover novel enzymes from plant-dwelling bacteria, preferably phytopathogens. Genome sequences of such bacteria show the presence of several putative cell wall degrading enzymes (Rubin, E. M. 2008. Genomics of cellulosic biofuels. Nature. 454: 841-845). Characterization of new enzymes with novel cell wall hydrolysing activities can lead to the formulation of enzyme mixtures, which can provide the possibility to completely degrade plant cell walls and be advantageous to the art.

X-ray crystal structures of certain plant cell wall degrading enzymes have shown presence of carbohydrate-binding modules (CBMs) that introduce an added feature of sugar binding for efficient substrate recognition in the context of polysaccharide-rich plant cell walls (Boraston, A. B., Bolam, D. N., Gilbert, H. J. and Davies, G. J. 2004. Carbohydrate-binding modules: fine-tuning polysaccharide recognition. Biochem. J. 382: 769-781). About 53 different families of CBMs are reported, out of which, structures are available for 30 families (Cantarel, B. L. et al. 2008. The Carbohydrate-Active EnZymes database (CAZy): an expert resource for Glycogenomics. Nucl. Acids Res. gkn663v1-gkn663). The current invention deals with a novel plant cell wall degrading enzyme containing a novel mode of carbohydrate binding wherein the enzyme can bind to both glucose and acyl chain moieties of an uncharacterized glycoside substrate in plant cell wall and this feature was evident only from its structural analysis and not from the sequence analysis. It would be advantageous to use such novel enzymes and their modifications thereof, for degradation of complex polysaccharide constituents of plant cell walls in biofuel production.

### OBJECT OF THE PRESENT INVENTION

The main object of the present invention is to provide variant of LipA that binds to the glucosyl- and acyl chain-region of its glycoside substrate in plant cell walls useful for biofuel production.

Yet another object is to use a novel mode of plant cell wall degradation activity of Xanthomonas oryzae pathovar oryzae esterase, LipA (and closely related proteins with similar mode of action), which involves recognition of glucose and acyl chain components in its plant substrate for plant cell wall degradation with the final goal of biofuel production.

### SUMMARY OF THE PRESENT INVENTION

The present invention relates to a novel esterase enzyme LipA and its variants that have recognition pockets for glucosyl and acyl chain, as detected using its X-ray crystal structure. This invention is based on the novel mode of enzymatic degradation of a glycoside substrate in the plant cell wall.

The said enzyme can increase the efficiency of the enzyme cocktails used for plant biomass for biofuel production. This invention also relates to a method of creating mutants at structure-based amino acid positions and saturation/random mutagenesis of the said enzyme to enhance the said activity.

Accordingly, the present invention provides variant of LipA that binds to the glucosyl- and acyl chain-region of its glycoside substrate in plant cell walls useful for biofuel production.

In an embodiment of the present invention a variant of LipA is having mutation in the region from 210 to 318 aminoacid of the native LipA protein.

In another embodiment of the present invention in a variant of LipA the mutations are selected from the group comprising of N228W, G231A, G231I, G231F and G221I.

In yet another embodiment of the present invention the enzyme is derived from organism of the genus Xanthomonas.

In yet another embodiment of the present invention, presence of a hydrophobic pocket confers extensive hydrophobic interaction of the acyl chain with the rest of the tunnel residues in a variant of LipA.

In still another embodiment of the present invention, it relates to use of LipA and its variant for plant cell wall degradation.

In still another embodiment of the present invention, it relates to use of LipA and its variant involving a unique tunnel for binding its glycoside substrate in plant cell walls useful for biofuel production, the said tunnel being capable of binding to an acyl chain and glycoside moiety.

In still another embodiment of the present invention, it relates to use of LipA and its variant conjunction with any one or any combination of cellulases, cellobiosidases, xylanases, feruloyl esterases, acetyl xylan esterases, pectin methyl esterases and any other reported class of cell wall degrading enzymes to improve the efficiency of enzyme mixes for biofuel production.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE ACCOMPANYING DRAWINGS

FIG. 1: Three-Dimensional Structure of LipA (a) Stereoview of the ligand-bound structure of LipA showing two bound molecules of β-octyl glucoside and the catalytic triad. The hydrolase domain is depicted in pink and the ligand-binding domain in green. The ligand molecules (yellow), S176, D336 and H377 are given in a stick representation.

FIG. 2: Topology diagram of LipA. The β-sheets are shown as arrows, α-helices as cylinders and 3₁₀ helices as rectangles. The a set of helices form the ligand-binding domain (green).

FIG. 3: Residues lining the BOG-bound tunnel are shown. Electron density of the two BOG molecules is a 2F_{obs}-F_{calc} map contoured at 1σ value. The amino acids are shown as yellow sticks. Active site S176 is highlighted. The wild-type LipA residues (grey) of the same region are superimposed.

FIG. 4: Important BOG1-specific interactions in the carbohydrate-binding pocket of LipA are main-chain mediated and shown as dashed lines with distance in Å.

FIG. 5: Multiple sequence alignment of LipA ligand-binding domain with homologous regions in other bacteria. Xanthomonas oryzicola (Xoryp20705); X. campestris pv. vesicatoria (XCV0536); X .axonopodis pv. citrii (XACO501); X. campestris pv. campestris str. ATCC 33913 (XCC2957 & XCC2374); Xylella fastidiosa 9a5c (XF0357, XF0358 & XF2151); Burkholderia phytofirmans (Bphyt4125); B. xenovorans (BxeB0552); Ralstonia metallidurans (Rmet5769); Polaromonas napthalenivorans (Pnap1828); Streptomyces avermitilis (SAV5844); Ideonella sp. (BAF64544) and Candida antarticus (2VEO). Residues marked in green are amino acids lining the tunnel and the red dots indicate the residues in the carbohydrate-binding pocket that were mutagenized. The black dots represent 39-45 aa inserts in the sequences of the corresponding proteins that are not shown in the figure.

FIG. 6: Superposition of the lid domains of LipA (yellow) and CalA (blue). BOG and PEG are shown as yellow and blue sticks respectively. Active site serines for LipA and CalA are shown in yellow and blue respectively.

FIG. 7: LipA exhibits BOG binding activity in vitro. Binding isotherm of LipA titrated against BOG using isothermal titration calorimetry. Top panel: raw titration curve; Bottom panel: Heats fitted by non-linear regressional curve fitting using one site binding model.

FIG. 8: Binding parameters and thermodynamics of BOG titration with wild-type LipA and mutants calculated using ITC

FIG. 9: LipA exhibits esterase activity. Presence of zone of clearance indicates LipA activity on short chain triacylglycerides (1) C4 (tributyrin) and (2) C6 (tricaproin) while no activity is seen on (3) C8 (tricaprylin). Holes punched to the right side contain LipA and the left side contain buffer.

FIG. 10: Tributyrin clearance activity of (1) Buffer (2) LipA (wild-type) (3) S176A (4) N228W (5) G231A (6) G231I (7) G231F (8) G221I. Plates were photographed after 2 hr of incubation at room temperature.

FIG. 11: LipA mutant proteins are deficient at induction of defense response associated programmed cell death in rice roots. Rice roots were treated with purified proteins (either wild-type LipA or mutants), stained with propidium iodide (PI) and examined under a confocal microscope to assess the extent of DNA fragmentation. The control buffer-treated roots (a) exhibit a prominent cell wall associated autofluorescence but no internalization of PI into the cells. Treatment with either wild-type LipA (b) or G221I (h) resulted in cell death (intake of PI) accompanied by dispersed intracellular staining which is indicative of nuclear fragmentation. No cell death is seen in roots treated with S176A (c); N228W (d); G231A (e); G231I (f) and G231F (g).

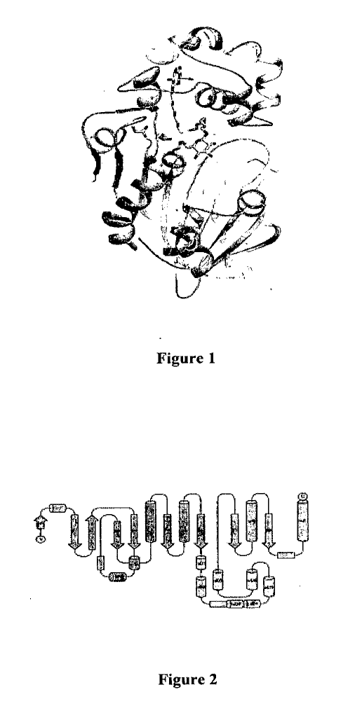

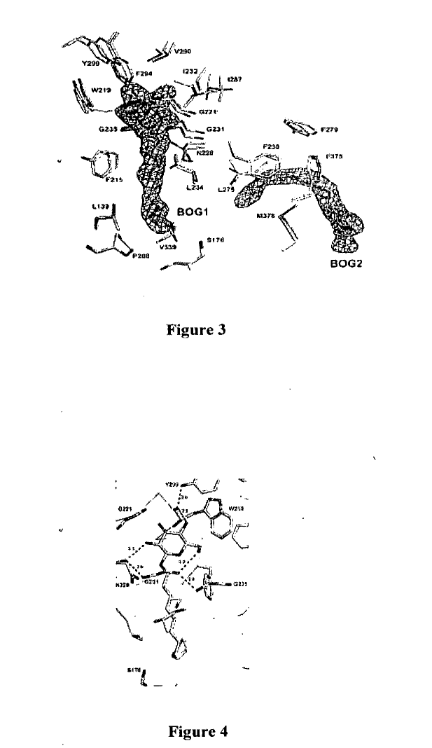

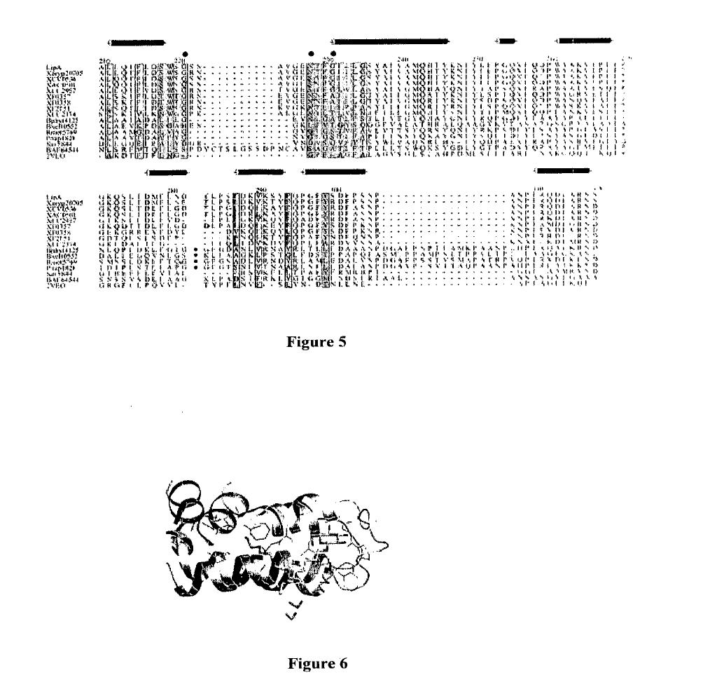

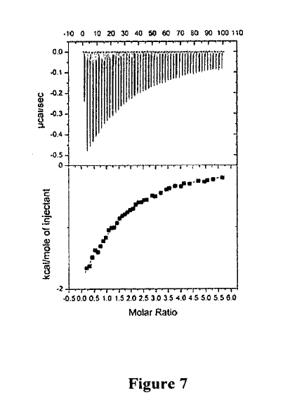

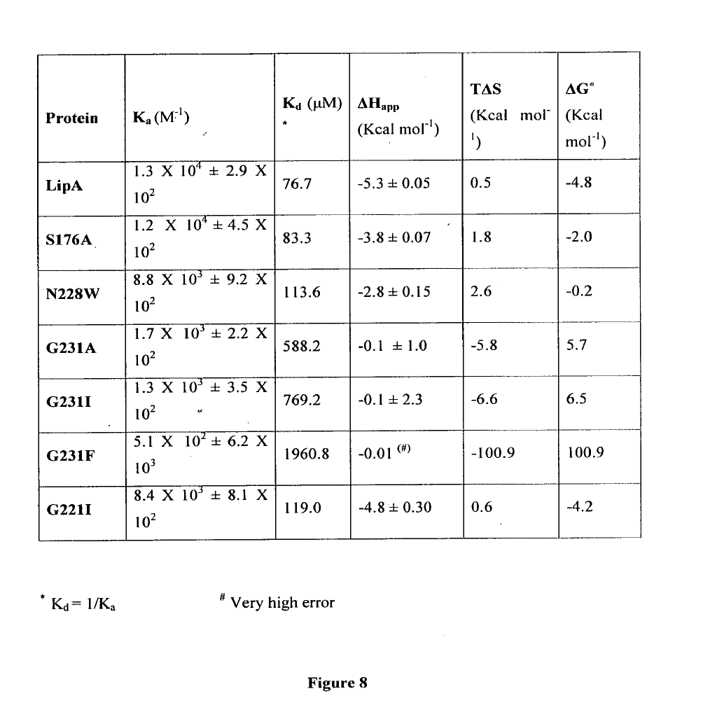

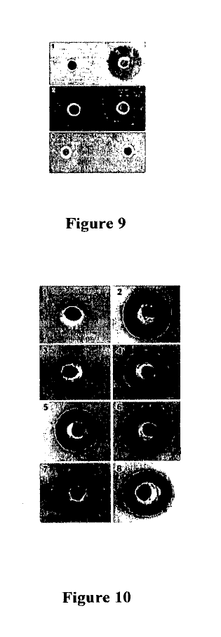

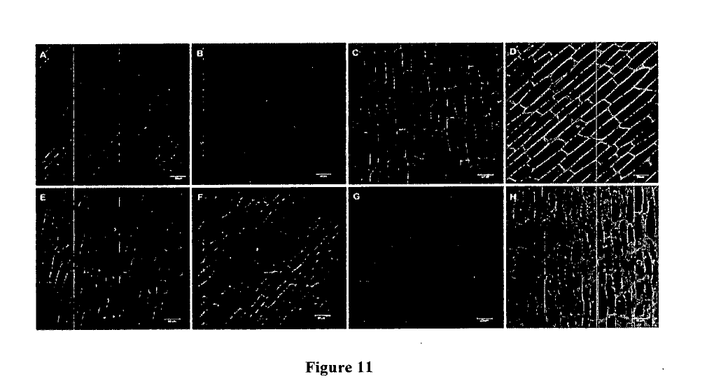

### DETAILED DESCRIPTION OF THE INVENTION

Biofuel is a non-polluting renewable source of energy and is emerging as a cheap and viable substitute to the present major source of energy, the fossil fuels. The most common starting material for biofuel production are the agricultural wastes that are rich in biomass and hence, carbon. The hydrolysis products, mostly sugars, are further processed to produce hydrocarbon rich compounds and alcohols that can be directly used as fuel. The degradation of plant biomass composed of chemically complex cell walls is the rate-limiting step in the whole process. Several hydrolytic enzymes of microbial origins have been found to degrade plant cell walls efficiently. Cellulolytic enzymes like endoglucanases, cellobiohydrolases, and beta-glucosidases hydrolyze the most abundant constituent, the cellulose fibrils. However, the efficiency of cell wall degradation is not good when only cellulose is hydrolysed. Enzyme cocktails containing xylanases, feruloyl esterases, acetyl xylan esterases, pectin methyl esterases etc. in addition to the cellulolytic enzymes increase the efficacy of biomass degradation. These additional enzymes act on the various types of cross-linked polymers that hold the cellulose fibrils in place and cleavage of the crosslinks or degradation of the polymers renders cellulose more susceptible to hydrolysis.

The exact chemical nature of all the plant cell walls is not yet fully understood. There might be several uncharacterized macromolecules or linkages in the plant cell walls. This also indicates that there will be several enzymes targeted specifically towards these novel chemical entities. This gives us an opportunity to improve upon the existing hydrolytic enzyme mixtures by addition of new classes of cell wall degrading enzymes.

Biochemical and structural studies on plant cell walls are restricted by the chemically complex nature of the constituent macromolecules. However, structural analyses of the cell wall degrading enzymes, on the other hand, is one of the most definitive ways of detecting novel structural features aiding in the hydrolysis of natural substrates. This can lead to identification of novel linkages and ligands for these enzymes also. The structure of LipA enzyme from Xanthomonas oryzae pathovar oryzae is an interesting outcome of this approach. LipA structure reveals that this enzyme is an α/β hydrolase fold protein with a 9-stranded central mixed beta-sheet surrounded by alpha helices in a typical hydrolase topology. The canonical catalytic triad residues Ser 176, His 377 and Asp 336 are positioned similar to several other hydrolases. The nucleophilic S176 lies on a ‘strand-turn-helix elbow’ forming a G-X-S-X-G motif that is conserved in hydrolases (Jaeger, K.-E., Dijkstra, B. W. & Reetz, M. T. Bacterial biocatalysts: Molecular biology, structure and biotechnological applications of lipases. Annu. Rev. Microbiol. 53, 315-351 (1999). The distinctive feature of LipA structure is the presence of a 108 amino acid domain present as an insertion between the β6 and β7, corresponding topologically to the lid domain insertion position of triacylglycerol lipases. This insertion domain consists of seven alpha helices with one 3₁₀ helix. The sequence identity of LipA with characterized hydrolase fold proteins is very low. Closest structural hit for the entire LipA is Candida antarticus lipase CalA (PDB code: 2VEO) (Ericsson, D. J. et al. X-ray structure of Candida antarcticus lipase A shows a novel lid structure and a likely mode of interfacial activation. J. Mol. Biol. 376, 109-119 (2008) and other homologs are mostly acyl-amino acid peptidases (PDB code:1VE6) and dipeptidyl peptidases, (PDB code: 2D5L) which do not possess a similar lid-like domain, and the structural similarity is limited to the hydrolase domain only.

We screened for putative ligands of LipA using detergents and fatty acid additives that could mimic its natural substrate(s) in planta using cocrystallization. LipA cocrystallized with the glycoside detergent β-octyl glucoside (BOG) in the same crystallization condition as that of the wild-type. The cocrystal structure solution at 2.1 Å resolution showed two molecules of bound BOG. One molecule (referred to as BOG1) has B-factors in the range of 18-23 Å² while the other molecule (BOG2) is loosely bound and has higher B-factors (45-50 Å²). The ligand acyl chains, placed very close to each other (6.9 Å), disclose a 30 Å ‘tunnel’ passing by the active site residues and ending very close to the outer surface of the protein. BOG2 glucose moiety hangs out of the tunnel facing the solvent. The proximity of BOG1 terminal methyl group with Ser 176 active site residue (3.8 Å) strengthens the idea that this tunnel could be involved in substrate binding. The lid-like domain may also not exhibit any domain motion with respect to the hydrolase-fold upon ligand binding, unlike large movements seen in conventional lid domains of lipases during interfacial activation (28). The glucoside moiety of BOG1 interacts with the main chain atoms of LipA at the extreme end of the tunnel, ˜15 Å away from the active site serine, in a pocket made of three glycines and a few other polar residues. The rest of the tunnel is lined with several hydrophobic residues that trace the tunnel from the entrance upto the sugar-binding pocket. Presence of this hydrophobic pocket suggests that the moderate specificity conferred by the few hydrogen bonds on the sugar moiety of the ligand is sustained by extensive hydrophobic interaction of the acyl chain with the rest of the tunnel residues. The structural identification of a novel mode of glycoside ligand binding with independent and overlapping recognition pockets for both carbohydrate and acyl chain components is the basis of our invention.

Structural superposition of LipA with CalA illustrates that the PEG molecule bound in CalA structure occupies a very different ligand-binding pocket. The analogous region in LipA is packed with hydrophobic amino acids and the PEG molecule will have substantial clashes with them. In addition, the CalA region that superimposes on the LipA carbohydrate-binding pocket is predominantly occupied by the main chain of a loop, indicating that there is no room for such a pocket in CalA. Therefore, it is clear that the pocket in LipA is unique in nature with a specific carbohydrate-binding site located far away from the solely acyl-binding pocket of CalA. This finding advocates for LipA-like proteins to be grouped as a novel class of cell wall degrading esterases.

Esterases and lipases form a large group of hydrolase fold proteins. Several insertions and deletions in the basic hydrolase scaffold are found in nature, each change evolving towards hydrolyzing esters found in the local habitats of the pertinent organisms, thereby altering the substrate-specificity. The lid domain of lipases is a specific adaptation for long chain triacylglycerols. Association of the catalytic hydrolase domain with additional non-lid non-catalytic domains for specialized substrate binding has been seen in other enzymes also. LipA structure reveals the presence of a large lid-like domain, which seems to have evolved for a plant-associated esterase function. It is important to note that despite the presence of a very large lid with several hydrophobic residues, LipA does not show interfacial activation and exhibits esterase activity. Low r.m.s. deviation among the main chain Ca and side chains of LipA in the ligand bound and the wild-type structures clearly suggest that this domain remains rigid both in the presence and absence of a ligand. The nature of a plant substrate for LipA can be an amphiphilic molecule with a glucose (or perhaps, xylose) moiety attached to a long (substituted) acyl chain (or aryl ring) of a length of 16-18 carbons (˜30 Å) with an ester bond situated ˜10 Å from the sugar ring. The natural substrate could belong to a long cross-linked chain of polysaccharides and glycosides.

The glycosyl recognition pocket has several interesting residues. The protein-sugar interaction is primarily mediated by main chains of Gly 231, Trp219, Ser218 and Asn228. The close proximity of Gly231 with the sugar ring of BOG suggests that the smallest replacement at this position would have a severe effect on LipA action and therefore, Gly 231 was mutated to Ala, Ile and Phe. We found that all the Gly 231 point mutants were deficient in BOG binding in vitro and deficient for function in planta, suggesting that this residue is indeed required for the structural integrity of the substrate-binding pocket. Sequence and structural analysis of all the proteins homologous to LipA indicated that the Gly231 evolved in only genus Xanthomonas. Even the closest relative of Xanthomonas, Xylella fasidiosa has a Gly to Ala/Ile substitution, indicating obliteration of the sugar-binding pocket. The residue Asn 228 was mutated to Trp to block the tunnel just below the carbohydrate-binding pocket, which would protrude into the acyl-chain binding region. The N228W mutation affects LipA function in planta indicating that it disrupts binding to the natural substrate. Gly221, Ile232, Tyr299, Gly235, Va1290, Ile287 and Phe215 are some of the residues that are necessary for the maintenance of the pocket. Upon superposition of β-D-galactose sugar ring on the β-D-glucose ring of BOG shows the residue Ile232 to be very important for rejection of galactose in the pocket. The hydrophobic acyl chain-recognition pocket is lined by the residues Leu234, Leu275, Phe230, Phe279, Phe375, Va1339, Leu139 and an interesting Met378.

In this patent, we describe a new class of plant cell wall degrading enzymes that uses a unique mode of glycoside substrate recognition by binding to both glucosyl- and acyl chain components. Neither this mode of substrate recognition nor any enzyme known to hydrolyse such a substrate is under use for plant cell wall degradation with an aim of biofuel production. The identification of this unique substrate binding came from the X-ray crystal structure analysis of the esterase LipA derived from Xanthomonas oryzae pathovar oryzae. We also describe a method for improving the existing plant cell wall degrading enzyme cocktails by addition of enzymes possessing the said mode of substrate recognition. We also describe the use of the LipA crystal structure for generation of mutants with enhanced plant cell wall degrading activity of the said enzyme.

### EXAMPLE 1

Overexpression and Purification of LipA Enzyme of X. oryzae Pathovar Oryzae

X. oryzae pv. oryzae strain BXO2001 has an insertion mutation in the lipA gene, disrupting the enzyme activity (Rajeshwari, R., Jha, G. and Sonti, R. V. 2005. Role of an in planta expressed xylanase of Xanthomonas oryzae pv. oryzae in promoting virulence on rice. Mol. Plant Microbe Interact 18: 830-837). Introduction of the lipA gene into BXO2001 in the broad host-range vector pHM1 (to create BXO2008) using standard cloning techniques in the art (Sambrook, J., E. F. Fritsch, and T. Maniatis. 1989. Molecular cloning: a laboratory manual, 2^{nd} ed. Cold Spring Harbor Laboratory Press, Cold Spring Harbor, N.Y.) not only restores the lipase activity but also results in the overproduction of lipase (Aparna, G., Chatterjee, A., Jha, G., Sonti, R. V. and Sankaranarayanan, R. 2007. Crystallization and preliminary crystallographic studies of LipA, a secretory lipase/esterase from Xanthomonas oryzae pv. oryzae. Acta Cryst. F63: 708-710). The overexpressed LipA is also secreted into the medium like the wild type LipA. Approximately 5 mg LipA is secreted into the medium by 1 litre wild-type X. oryzae pv. oryzae. The BXO2008 strain secretes approximately 25-30 mg LipA per litre of culture. LipA can be purified from the BXO2008 culture supernatant using cation-exchange chromatography using 10 mM potassium phosphate buffer pH 6.0 (Jha, G., Rajeshwari, R. and Sonti, R. V. 2007. Functional interplay between two Xanthomonas oryzae pv. oryzae secretion systems in modulating virulence on rice. Mol. Plant Microbe Interact. 20: 31-40). The peaks of LipA can be further purified using a 24 ml Superose-12 gel-filtration column (GE Pharmacia, USA) pre-equilibrated with 10 mM potassium phosphate buffer pH 6.0. The purified protein can be dialyzed against 20 mM Tris-HCl pH 7.5, 20 mM NaCl and concentrated upto 5 mg ml⁻¹ using al0 kDa Amicon Ultra-15 filtration device (Millipore, USA). Protein concentration can be determined using Bradford's reagent (Bradford, M. M. 1976. A rapid and sensitive method for quantitation of microgram quantities of protein utilizing the principle of protein-dye binding. Anal. Biochem. 72:248-254).

X oryzae pv. oryzae strains are grown at 301 K in peptone-sucrose (PS) medium (Tsuchiya, K., T. W. Mew, and S. Wakimoto. 1982. Bacteriological and pathological characteristics of wild type and induced mutants of Xanthomonas campestris pv. oryzae. Phytopathology 72:43-46). The antibiotic spectinomycin (50 mg ml⁻¹) is to be used for growing X. oryzae pv. oryzae strains containing pHM1 vector.

### EXAMPLE 2

Overexpression and Purification of LipA Enzyme in Non-host E. coli

The plasmid pET28b (Novagen, USA) can be purified from cultures of Escherichia coli by the alkaline lysis method (13). PCR (Polymerase Chain Reaction) primers for the lipA gene are designed, by methods familiar to those well versed in the art, to incorporate the sites for NdeI and EcoRI. PCR is performed with Phusion Polymerase (Finnzymes, USA) using X. oryzae pv. oryzae genomic DNA as template to obtain a single fragment of approximately 1200 bp. This fragment can be digested with restriction enzymes NdeI and EcoRI obtained from NEB. This fragment is cloned into the NdeI and EcoRI sites of pET28b using standard procedures as described (Sambrook, J., E. F. Fritsch, and T. Maniatis. 1989. Molecular cloning: a laboratory manual, 2^{nd} ed. Cold Spring Harbor Laboratory Press, Cold Spring Harbor, N.Y.) to generate a fusion of the lipA with a 6X-His tag that is encoded on pET28b. Exponentially growing E. coli cultures containing this recombinant lipA plasmid are induced with IPTG (Isopropyl thiogalactoside; a potent inducer of the promoter to which lipA gene is fused) to produce large amounts of the lipA-His tag fusion protein. The uninduced plasmid would not produce the fusion protein. The cells can be sonicated and the overexpressed fusion protein purified using Ni-NTA packed column as per manufacturer's (Qiagen) instructions. LipA protein can be obtained by cleaving the fusion protein with FactorXa protease.

### EXAMPLE 3

Crystallization and Data Collection of LipA Enzyme of Xanthomonas oryzae Pathovar Oryzae

Crystallization drops can be set up using the hanging-drop vapour-diffusion method by mixing equal volumes (2 ml) of protein solution and reservoir solution at 298 K. Well-diffracting crystals are obtained in conditions with 11% PEG 6000 as precipitant and 0.10 M MES buffer pH 6.7. MAR Research MAR345dtb image-plate detector and Cu Kα X-rays of wavelength 1.54 Å generated by a RigakuRU-H3R rotating-anode generator can be used to collect X-ray diffraction data. The crystals can be mounted on a nylon loop and flash-cooled in a nitrogen gas stream at 100 K using an Oxford Cryostream system. 15% glycerol in the mother liquor is required for cryoprotection of the crystals. Data is to be collected with an oscillation angle of 0.5° and an exposure time of 600 s for each image. A total of 180° of oscillation data is to be collected for the crystals. Indexing, scaling and merging of the data can be performed using DENZO and SCALEPACK (Otwinowski, Z. and Minor, W. 1997. Processing of X-ray diffraction data collected in oscillation mode. Methods Enzymol. 276: 307-326).

### EXAMPLE 4

Structure Solution of LipA Enzyme of Xanthomonas oryzae Pathovar Oryzae

LipA structure was solved using Multiple Isomorphous Replacement method because this protein has no homologs with sequence identity more than 20% and our attempts to solve LipA structure using Molecular Replacement method failed. Isomorphous LipA crystals soaked in heavy atoms (Hampton, USA) like 10 mM K₂PtCl₂ for 2-15 min and 10 mM Sm (NO₃)₂ soaks of 20-25 min are used for heavy atom substitution. Long soaks of 1 hr can be used for the Hg salt thimerosol to obtain substitution. The heavy atom soaked crystal data can also be collected like the native non-soaked crystals as mentioned in Example 2. The SOLVE run (Terwilliger, T. C. & Berendzen, J. 1999. Automated MAD & MIR structure solution. Acta Cryst. D55: 849-861) with 4 Pt, 1 Sm and 1 Hg data set gives a figure of merit 0.45 and Z score 40. Phase extension and improvement can be performed using DM (Computational Collaborative Project, Number 4, Program suite). RESOLVE (21) can build only 150 alanines and 30 side chain residues out of 397 expected amino acids. Iterative rounds of chain building using the experimental map for the rest of the 217 residues and RESOLVE runs for localised loop building are to be performed. The structure can be refined using CNS. Structure visualization and a part of model building can be done using the software O (Terwilliger, T. C. 2003. Automated main-chain model building by template matching and iterative fragment extension. Acta Cryst. D59: 38-44).

### EXAMPLE 5

Analysis of the LipA Enzyme Structure

NCBI BLAST server may be used for identifying sequence homologs of LipA from several other organisms (Altschul, S. F., T. L. Madden. A. A. Schaffer, J. Zhang, W. Mifler and D. J. Lipman, 1997. Gapped BLAST and PSI-BLAST; a new generation of protein database search programs. Nucleic Acids Res. 25:3389-3402). ClustalW (EBI server) can be used to generate multiple sequence alignments among the sequence homologs and phylogenetic trees and bootstrap analysis may be prepared using MEGA v.4 (Tamura, K., Dudley, J., Nei, M. & Kumar, S. 2007. MEGA4: Molecular Evolutionary Genetics Analysis (MEGA) software version 4.0. Mol. Biol. Evol. 24: 1596-1599). Modelling of LipA homologs can be done using Modeller9v4 software (Marti-Renom, M. A. et al. 2000. Comparative protein structure modeling of genes and genomes. Annu. Rev. Biophys. Biomol. Struct. 29: 291-325). This exercise would help identify all the other organisms having LipA-like enzyme activity using the novel mode of substrate binding and. DALI server (Sali, A. & Blundell, T. L. 1993. Comparative protein modelling by satisfaction of spatial restraints. J. Mol. Biol. 234: 779-815) can be used for structural homology searches. However, there are no structural homologs using LipA-like mode of substrate binding.

Upon soaking LipA wild-type crystals with17.5 mM of β-octyl glucoside, the glycoside ligand binds to the enzyme. The structure of this ligand-bound LipA can be solved using Molecular Replacement method (Vagin, A. and Teplyakov. A (1997). MOLREP: an Automated Program for Molecular Replacement. J. Appl. Cryst. 30, 1022-1025). The ligand-bound structure can be analysed to identify the novel substrate-binding tunnel that has the two pockets for glycosyl and acyl chain binding. This tunnel can be compared to proteins in other organisms by analysing the various structures in structure visualisation softwares like O to detect whether the homologs of LipA also bind to the substrate in a similar manner. The LipA homologs from genus Xanthomonas show the presence of a similar mode of substrate binding despite infecting very diverse range of hosts (Leyns, F., M. DeCleene, J. G. Swings, and J. Deley. 1984. The host range of the genus Xanthomonas. Bot. Rev. 50:308-356) indicating that this enzyme is required for plant cell wall degrading enzyme activity in a wide variety of plants.

### EXAMPLE 6

Site-Directed Mutagenesis and Purification of Mutant Variants of LipA

The lipA gene can be cloned into pBSKS plasmid (Stratagene, USA) from the pHM1clone for reduction of the total size of the vector to be handled for mutagenesis. Site-directed mutagenesis can be performed using the QuickChange site-directed mutagenesis kit (Stratagene, USA). The mutant lipA genes then has to be excised as KpnI-HindIII fragments, cloned back into the multi-cloning site of broad-host range vector pHM1 and transformed into X. oryzae pv. oryzae strain BXO2001 that has an insertion mutation in the lipA gene. Presence of the lipA point mutations has to be confirmed by sequencing of the LipA gene from each strain. Expression of mutant LipA proteins can be confirmed using rabbit polyclonal anti-LipA antibodies. X. oryzae pv. oryzae strain BXO2008 is used as a source of wild-type LipA. The LipA mutant proteins can be purified to homogeneity using the protocol in Example 1.

Site-directed mutagenesis of Gly 231, Trp219, Ser218, Asn228, Gly221, Ile232, Tyr299, Gly235, Va1290, Ile287 and Phe215 in the glucose-binding pocket can be performed. Conversion of these residues individually or in combinations will lead to variations in the size of the pocket and thus, the sugar specificity or affinity. The hydrophobic acyl chain-recognition pocket residues Leu234, Leu275, Phe230, Phe279, Phe375, Va1339, Leu139 and Met378 can also be subjected to site-directed mutagenesis to lead to altered specificity of LipA substrates.

DNA sequencing can be performed by using Applied Biosystems ABI DNA sequencer according to the protocol in the ABI Dye Terminator Cycle Sequencing kit.

### EXAMPLE 7

Site Saturation Mutagenesis and Purification of Mutant Variants of LipA

Every residue mentioned in Example 1 can be converted into all 20 amino acids by site directed mutagenesis using 32-fold degenerate oligonucleotide primers (Short, J. M. (2001) Saturation mutagenesis in directed evolution. U.S. Pat. No. 6,171,820). The primers randomize each codon and have the common structure X₂₀ NN(G/T)X20. PCR reaction is performed using Phusion polymerase (Finnzymes) as per very well known protocols in the art. The reaction mix is digested with 10 U of DpnI at 37° C. for 1 hour to digest the methylated template. 10 μl reaction mix can be used to transform 40 μl of DH5α cells and the entire transformation mix plated on a large LB-Ampicillin-100 plate.

Screening is performed by sequencing using full-length LipA primers to assess the type of mutation at the particular site. DNA sequencing can be performed by using Applied Biosystems ABI DNA sequencer according to the protocol in the ABI Dye Terminator Cycle Sequencing kit. The LipA mutant proteins can be purified to homogeneity using the protocol in Example 1.

### EXAMPLE 8

Random Mutagenesis and Purification of Mutant Variants of LipA Using the Entire Coding region of LipA

To randomly mutate lipA gene, error-prone polymerase chain reaction (PCR) mutagenesis can be performed using a slightly modified version of the method of Leung et al. (Leung, D. W., Chen, E. and Goeddel, D. V. (1989) Technique, 1, 11-15). The pHM1-lipA plasmid can be treated with 12 M formic acid for 20 min. at room temperature. The resulting lipase encoding gene is amplified from the formic acid treated plasmid using PCR with Gene Taq DNA polymerase (Nippon gene, Tokyo) under mutagenic conditions (0.5 mM MnCl₂ and ⅕ the normal amount of ATP. The randomly mutated lipA genes were digested with HindIII and KpnI and then ligated into pHM1. The ligates were amplified in Escherichia coli. The recovered plasmids can be called randomly mutated lipA gene library. DNA sequencing can be performed by using Applied Biosystems ABI DNA sequencer according to the protocol in the ABI Dye Terminator Cycle Sequencing kit. The LipA mutant proteins can be purified to homogeneity using the protocol in Example 1.

### EXAMPLE 9

Substrate Clearance Assay to Confirm that Mutant LipA Proteins have Enzyme Activity

LipA shows clearance of tributyrin substrate. 50 μl of 0.5 mg ml⁻¹ of purified wild-type and mutant LipA proteins are to be added to the wells cut into each substrate plate and the zone of clearance assays is performed at room temperature.

### EXAMPLE 10

Preparation of an Enzyme Mixture to Degrade Plant Lignocellulosic Biomass for Biofuel Production:

The constituents of degradative enzymes include any one or any combination of the following with LipA, but are not limited to:
1. Cellulolytic enzymes: Endoglucanase (cellulase), cellobiohydrolase, beta-glucosidase, endo-beta-1,3(4)-glucanase, glucohydrolase.
2. Xylolytic enzymes: Xyloglucanase, xylanase, xylosidase, alpha-arabinofuranosidase, alpha-glucuronidase, acetyl xylan esterase, xylogalacturonosidase, xylogalacturonase
3. Other polysaccharide-active enzymes: Mannanase, mannosidase, alpha-galactosidase, mannan acetyl esterase, galactanase, arabinanase, polygalacturonases, alpha-arabinofuranosidase, beta-galactosidase, galactanase, arabinanase, alpha-arabinofuranosidase, rhamnogalacturonase. rhamnogalacturonan lyase, rhamnogalacturonan acetyl esterase, rhamnogalacturonan lyase
4. Pectinolytic enzymes: Pectate lyase, pectin lyase, pectate lyase, pectin acetyl esterase, pectin methyl esterase, lignin peroxidases, lignin peroxidases and manganese-dependent peroxidases, and laccases.
5. Common commercially available enzymes: Celluclast, Novozym188, Celluzyme Cereflo, UltraFlo, Shearzyme, Biofeed Wheat, Bio-feed Plus L, Viscozyme, Pentopan Mono BG, Pulpzyme HC, Lecitase, Lipolase, Lipex, Alcalase, Savinase and Neutrase (Novozymes); Laminex, Spezyme CP (Genencor Int.); Rohament 7069 W (Rohm GMBH) can be used according to the manufacturers' advise.

Cocktails of several or all the above-mentioned enzymes can be prepared in different concentrations depending on the weight of the biomass, ranging from 0.1-2.0% of the solid weight.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated by reference in their entireties.

1. A variant of LipA capable of binding to a glucosyl- and an acyl chain-region of a glycoside substrate in plant cell walls, wherein the variant of LipA is useful for biofuel production.

2. The variant of LipA as claimed in claim 1 having a mutation in the region from amino acid residue 210 to amino acid residue 318 of a native LipA protein of SEQ ID No: 1.

3. The variant of LipA as claimed in claim 1, wherein said mutations is selected from the group consisting of N228W (SEQ ID No: 2), G231A (SEQ ID No: 3), G231I (SEQ ID No: 4), G231F (SEQ ID No: 5) and G221I (SEQ ID No: 6).

4. The variant of LipA as claimed in claim 1 wherein the variant of LipA is derived from an organism of the genus Xanthomonas.

5. The variant of LipA as claimed in claim 1, 2, 3, or 4, further comprising a hydrophobic pocket and a unique tunnel for binding the glycoside substrate, said tunnel comprising tunnel residues, wherein presence of hydrophobic pocket is capable of conferring extensive hydrophobic interaction of the acyl chain region with the tunnel residues.

6. A method of using of the LipA variant as claimed in claim 1, 2, 3, 4, or 5 for plant cell wall degradation.

7. A method of using of the LipA variant as claimed in claim 6, comprising the step of binding a unique tunnel with the glycoside substrate in plant cell walls useful for biofuel production, said tunnel being capable of binding to the glucosyl and the acyl chain region of the glycoside substrate.

8. A method of using of the LipA and its variant as claimed in claim 6 or 7, further comprising the step of using cellulases, cellobiosidases, xylanases, feruloyl esterases, acetyl xylan esterases, pectin methyl esterases and any other reported class of cell wall degrading enzymes or a combination thereof to improve the efficiency of enzyme mixes for biofuel production.
